(19) United States
(12) Patent Application Publication
(10) Pub. No.: US 2025/0140426 A1
(43) Pub. Date: May 1, 2025

(54) INFORMATION SHARING SYSTEM AND RECORDING MEDIUM

(71) Applicant: Konica Minolta, Inc., Tokyo (JP)

(72) Inventor: Moeko HAGIWARA, Ichikawa-shi (JP)

(21) Appl. No.: 18/918,305

(22) Filed: Oct. 17, 2024

(30) Foreign Application Priority Data
Oct. 26, 2023 (JP) 2023-183992

(51) Int. Cl. G16H 80/00; H04L 67/06; H04L 67/60

(52) U.S. Cl. CPC G16H 80/00; H04L 67/06; H04L 67/60

(57) ABSTRACT

An information sharing system includes a first hardware processor that transmits a data sharing request from a medical facility terminal used by a medical professional to a user terminal used by a patient, and a second hardware processor that accepts the sharing request transmitted from the first hardware processor, wherein the second hardware processor, upon acceptance of the sharing request, causes a display included in the user terminal to display an approver for accepting approval for sharing the data with the medical facility terminal, and upon acceptance of the approval for sharing the data with the medical facility terminal by the approver, causes the data to be shared with the medical facility terminal.

### CROSS-REFERENCE TO RELATED APPLICATIONS

The entire disclosure of Japanese Patent Application No. 2023-183992 filed on Oct. 26, 2023 is incorporated herein by reference in its entirety.

### BACKGROUND OF THE INVENTION

### Technical Field

The present invention relates to an information sharing system and a recording medium.

### Description of Related Art

A patient with a chronic disease may be required not only to be examined at the time of a visit to a medical institution, but also to record the situation between visits by the patient himself/herself and report the recorded situation to a doctor at the time of visit.

In recent years, an application in which a patient records his/her own vital information and situations of exercise, meals, medications, and the like has become common. When the patient records these pieces of information, it is possible to prompt the patient to improve adherence and change behavior. A mechanism has also been proposed in which a doctor can view these pieces of information recorded by the patient.

In this regard, the following system is disclosed in JP 2003-319912A. To be specific, the patient registers, with the server, the vital information measured by himself/herself for the items instructed by the doctor. When the vital information is registered with the server, the system notifies the medical institution of the fact, and the doctor views the vital information of the patient.

Japanese Patent No. 7256490 discloses a system in which a doctor presets a recording item set necessary for a patient, and the patient inputs the content specified in the recording item set.

### SUMMARY OF THE INVENTION

However, the inventions of JP 2003-319912A and Japanese Patent No. 7256490 have problems in that the patient cannot select the content of the recording item set by himself/herself, and cannot record his/her situation from a time when the patient is healthy and show the record to a doctor in response to a request from the doctor.

An object of the present invention is to provide an information sharing system and a recording medium that allow a doctor to appropriately view data of a patient on the basis of the patient's intention.

To achieve at least one of the abovementioned objects, according to another aspect of the present invention, an information sharing system reflecting one aspect of the present invention includes:
a first hardware processor that transmits a data sharing request from a medical facility terminal used by a medical professional to a user terminal used by a patient; and
a second hardware processor that accepts the sharing request transmitted from the first hardware processor, wherein
the second hardware processor, upon acceptance of the sharing request, causes a display included in the user terminal to display an approver for accepting approval for sharing the data with the medical facility terminal, and upon acceptance of the approval for sharing the data with the medical facility terminal by the approver, causes the data to be shared with the medical facility terminal.

To achieve at least one of the abovementioned objects, according to another aspect of the present invention, a recording medium reflecting one aspect of the present invention is a non-transitory computer readable recording medium storing a program causing a computer of the information sharing system to perform:
accepting a data sharing request from a medical facility terminal used by a medical professional to a user terminal used by a patient;
displaying an approver for accepting approval for sharing the data with the medical facility terminal on a display included in the user terminal upon acceptance of the sharing request; and
sharing the data with the medical facility terminal upon acceptance of the approval for sharing the data with the medical facility terminal by the approver.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention, wherein:

FIG. 1 is a system configuration diagram of an information sharing system of the present embodiment;

FIG. 2 is a block diagram illustrating a functional configuration of an information management server;

FIG. 3 is a block diagram illustrating a functional configuration of a user terminal;

FIG. 4 is a block diagram illustrating a functional configuration of a medical facility terminal;

FIG. 5 is a ladder chart illustrating a first information sharing process;

FIG. 6 is a diagram illustrating an example of a patient list screen;

FIG. 7 is a diagram illustrating an example of an approval screen;

FIG. 8 is a diagram illustrating an example of a display screen; and

FIG. 9 is a ladder chart illustrating a second information sharing process.

### DETAILED DESCRIPTION

Hereinafter, one or more embodiments of the present invention will be described with reference to the drawings. However, the scope of the invention is not limited to the disclosed embodiments.

### [Configuration of Information Sharing System]

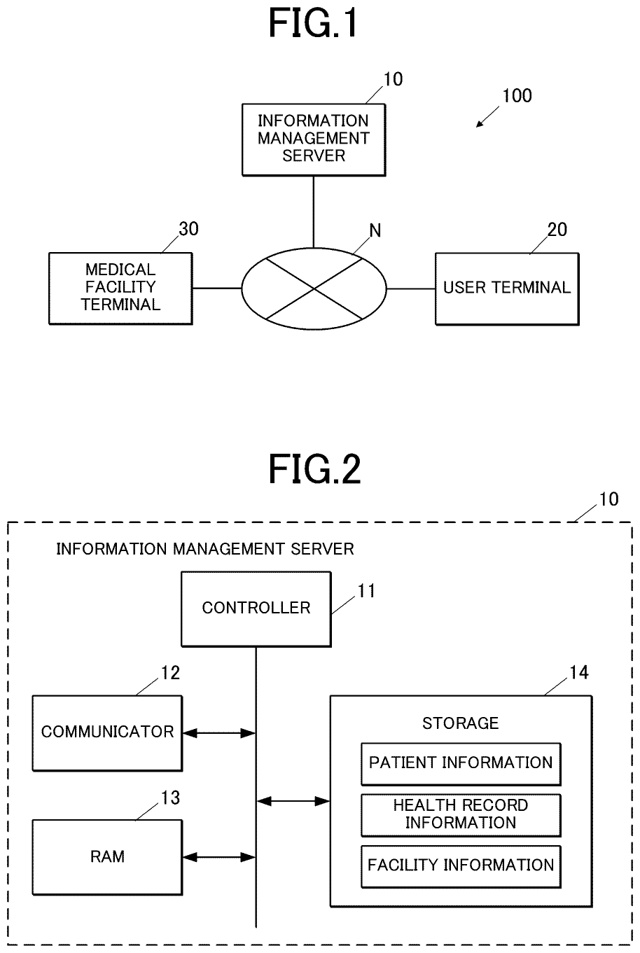

FIG. 1 illustrates a system configuration of an information sharing system 100.

As illustrated in FIG. 1, the information sharing system 100 includes an information management server 10, a user terminal 20, and a medical facility terminal 30.

The information management server 10 is connected to the user terminal 20 and the medical facility terminal 30 through a communication network N such as the Internet in a data communicable manner.

The information management server 10 is a device that accumulates, manages, and processes information on the medical care of a patient registered from the user terminal 20 and the medical facility terminal 30.

The information management server 10 is a cloud server provided in a cloud environment or a local server. For example, the information management server 10 is a personal health record (PHR) server.

The user terminal 20 is a terminal device such as a smartphone used by a user who is a patient.

The number of user terminals 20 is not limited to the example illustrated in FIG. 1.

The medical facility terminal 30 is a computer device such as a personal computer (PC) or a tablet terminal which is used by a medical professional (a doctor, a nurse, or the like) belonging to a medical facility.

The medical facility terminal 30 is, for example, a PHR management terminal that manages PHR information.

### [Configuration of Information Management Server]

FIG. 2 illustrates a functional configuration of the information management server 10.

As illustrated in FIG. 2, the information management server 10 includes a controller 11 (i.e., a second hardware processor), a communicator 12, a RAM 13, a storage 14, and the like, and these parts in the information management server 10 are connected to each other via a bus.

The controller 11 includes a central processing unit (CPU) and the like, and comprehensively controls a processing operation of each part in the information management server 10. To be specific, the CPU reads various processing programs stored in the storage 14 to load the programs into the RAM 13, and performs various processes in cooperation with the programs.

The communicator 12 includes a network interface or the like, and transmits and receives data to and from an external device connected through the communication network N.

The RAM 13 forms a work area for temporarily storing various types of information read from the storage 14 in the various processes executed and controlled by the controller 11. The various types of information include various programs, input data, output data, parameters, and the like.

The storage 14 includes a hard disk drive (HDD), a nonvolatile semiconductor memory, or the like.

The storage 14 stores various processing programs, parameters and files necessary for executing the programs, and the like.

The storage 14 stores patient information of a patient.

The patient information is information on the patient. The patient information includes a patient ID, a name of the patient, a gender of the patient, a date of birth of the patient, an age of the patient, an address and a telephone number of the patient, and the like.

The storage 14 stores health record information of a patient for each piece of information for identifying the patient (patient identification information).

The patient identification information is, for example, a patient ID. The patient ID is the same as a patient ID included in the patient information.

The health record information is information in which the health of the patient is recorded. The health record information includes, for example, vital information, medication information, mood information, and measurement results of a weight or the like. The health record information is, for example, continuously recorded information. The health record information is information for grasping a trend in health.

The vital information is information that the patient inputs via the user terminal 20. Alternatively, the vital information is information automatically taken into the user terminal 20 from a vital measurement device that measures the vital information.

The vital information includes, for example, a blood glucose level, a blood pressure, a heart rate, a body temperature, and a weight.

The medication information is information that the patient inputs via the user terminal 20.

The medication information is information on a patient's medication.

The medication information includes information indicating a result selected by the patient from among “has already taken”, “partially has taken”, and “has not taken” the medicine.

The mood information is information that the patient inputs via the user terminal 20.

The mood information is information on the mood of the patient.

The mood information includes information indicating a result selected by the patient from among “good”, “normal”, “bad”, “very bad”, “not selected”, and the like, for the mood.

The measurement result of the weight or the like is information that the patient inputs via the user terminal 20. Alternatively, the measurement result of the weight or the like is information that is automatically taken into the user terminal 20 from a measurement device that measures the weight or the like.

The storage 14 stores facility information of a medical facility.

The facility information is information on a medical facility. The facility information includes a facility ID of the medical facility, a facility name of the medical facility, an address and a telephone number of the medical facility, and the like.

### [Configuration of User Terminal]

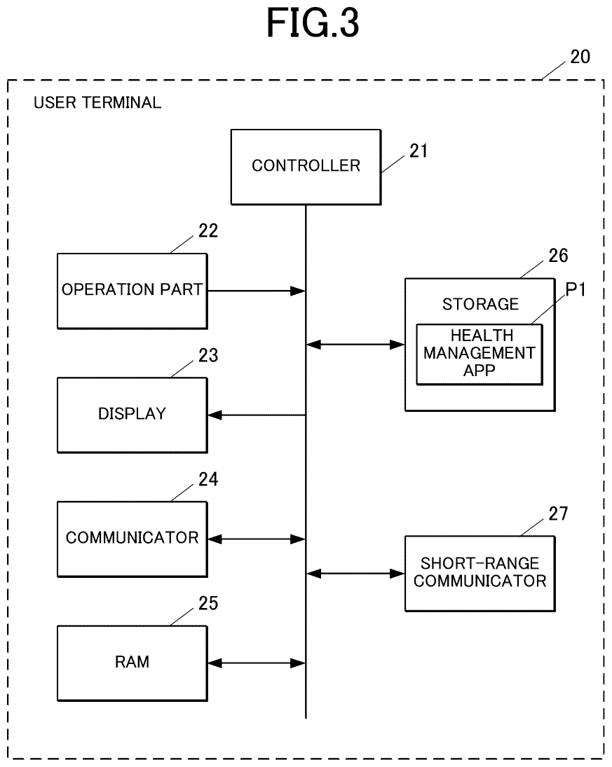

FIG. 3 illustrates a functional configuration of the user terminal 20.

As illustrated in FIG. 3, the user terminal 20 includes a controller 21 (i.e., a third hardware processor), an operation part 22, a display 23, a communicator 24, a RAM 25, a storage 26, a short-range communicator 27, and the like. These parts in the user terminal 20 are connected via a bus.

The controller 21 includes a CPU and the like, and comprehensively controls a processing operation of each part in the user terminal 20. To be specific, the CPU reads various processing programs stored in the storage 26 in response to an operation signal input from the operation part 22 or an instruction signal received by the communicator 24. Next, the CPU loads the read various processing programs into RAM 25, and performs various processes in cooperation with the programs.

The operation part 22 includes operation keys such as a power key for turning on and off the power, a touch screen laid on the display 23, and the like.

The operation part 22 outputs, to the controller 21, an operation signal corresponding to each of the operation keys, and an operation signal corresponding to a position touched by an operator's finger or the like.

The display 23 includes a liquid crystal display (LCD) or the like.

The display 23 displays various screens in accordance with an instruction of a display signal input from the controller 21.

The communicator 24 includes a network interface or the like, and transmits and receives data to and from an external device connected through the communication network N.

The communicator 24 performs wireless communication using a mobile phone line or the like, and transmits and receives data to and from an external device connected through the communication network N.

The RAM 25 forms a work area for temporarily storing various types of information read from the storage 26 in the various processes executed and controlled by the controller 21. The various types of information include various programs, input data, output data, parameters, and the like.

The storage 26 includes an HDD, a nonvolatile semiconductor memory or the like, and stores various processing programs and parameters and files necessary for executing the programs.

The storage 26 stores a program of a health management application (hereinafter, referred to as a “health management app”) P1. The patient uses the information-sharing service provided by the information management server 10 via the health management app P1. To be specific, the patient registers the health record information with the information management server 10 on the health management app P1 in the user device 20. The patient views the health record information managed by the information management server 10 on the health management app P1 in the user device 20.

The controller 21 transmits the health record information input from the operation part 22 or the short-range communicator 27 to the information management server 10 via the communicator 24.

For example, the controller 21 acquires the vital information from the vital measurement device via the short-range communicator 27. The controller 21 acquires, via the short-range communicator 27, a measurement result of the weight or the like from the measurement device that measures the weight or the like. Alternatively, the controller 21 acquires a measurement result of vital information, medication information, mood information, weight, or the like input from the operation part 22 by the patient.

As a cooperation destination of the health management app P1, a medical facility app P2 (described later) stored in the storage 36 in the medical facility terminal 30 is set in advance. The health management app P1 operates in cooperation with the medical facility app P2.

The short-range communicator 27 performs data communication with an external device via an antenna for transmission and reception in near field communication (NFC), Bluetooth® communication, or the like. The short-range communicator 27 receives, for example, the vital information from the vital measurement device. The short-range communicator 27 receives, for example, a measurement result of the weight or the like from the measurement device that measures the weight or the like.

### [Configuration of Medical Information Terminal]

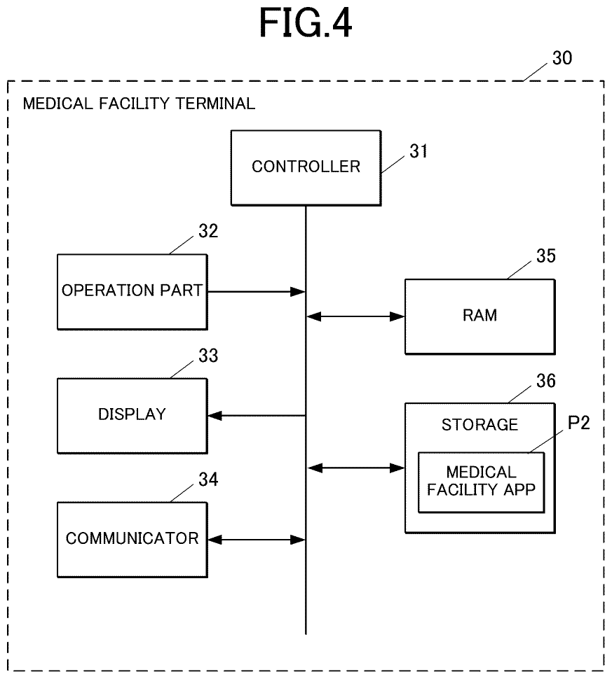

FIG. 4 illustrates a functional configuration of the medical facility terminal 30.

As illustrated in FIG. 4, the medical facility terminal 30 includes a controller 31 (i.e., a first hardware processor), an operation part 32, a display 33, a communicator 34, a RAM 35, and a storage 36. These units in the medical facility terminal 30 are connected via a bus.

Since the controller 31, the display 33, and the RAM 35 in the medical facility terminal 30 are the same as the controller 21, the display 23, and the RAM 25 in the user terminal 20, respectively, the description thereof will be omitted.

The operation part 32 includes a keyboard having cursor keys, character input keys, various function keys, and the like, and a pointing device such as a mouse.

The operation part 32 outputs, to the controller 31, an operation signal input by a key operation on the keyboard or a mouse operation.

When the medical facility terminal 30 is a tablet terminal or the like, the operation part 32 includes operation keys such as a power key for turning on and off the power, a touch screen laid on the display 33, and the like. The operation part 32 in this case outputs, to the controller 31, an operation signal corresponding to each of the operation keys, and an operation signal corresponding to a position touched by an operator's finger or the like.

The communicator 34 includes a network interface or the like, and transmits and receives data to and from an external device connected through the communication network N.

The storage 36 includes an HDD, a nonvolatile semiconductor memory or the like, and stores various processing programs and parameters and files necessary for executing the programs.

The storage 36 stores a program of the medical facility application (hereinafter, referred to as a “medical facility app”) P2. A medical professional such as a doctor uses the information sharing service provided by the information management server 10 via the medical facility app P2. To be specific, the medical professional views the health record information of the patient managed by the information management server 10 on the medical facility app P2 in the medical facility terminal 30.

### [Operation of Information Sharing System]

Next, the operation in the information sharing system 100 will be described.

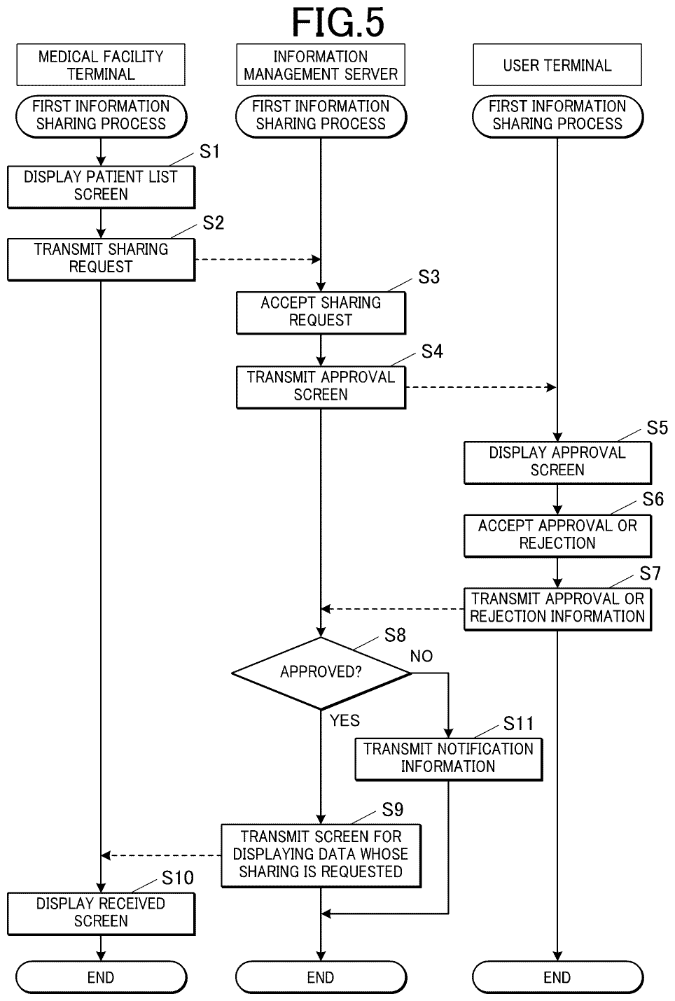

Each device included in the information sharing system 100 executes a first information sharing process illustrated in FIG. 5.

### (First Information Sharing Process)

The controller 31 in the medical facility terminal 30 activates the medical facility app P2 on the basis of an operation performed by a medical professional via the operation part 32. When the medical facility app P2 is activated, the controller 31 receives the patient list screen generated by the controller 11 from the information management server 10. Next, the controller 31 displays the received patient list screen on the display 33 (step S1).

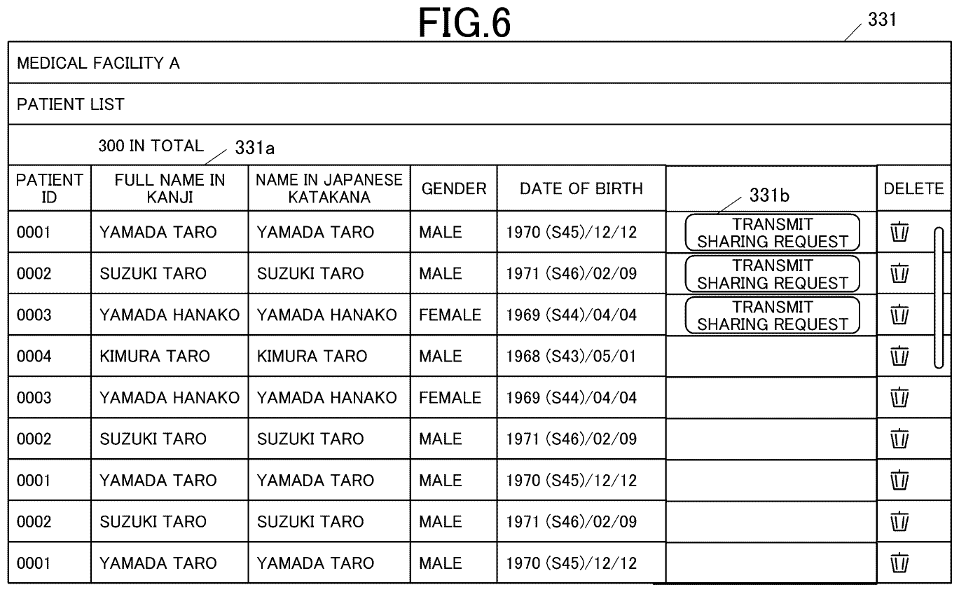

FIG. 6 illustrates an example of a patient list screen 331.

In the example illustrated in FIG. 6, the controller 11 causes a patient list 331a, which is a list of patient information stored in the storage 14 in the information management server 10, to be displayed on the patient list screen 331.

The controller 11 causes a patient ID, a patient name, a name of the patient, a gender of the patient, a date of birth of the patient, and the like, which are included in the patient information, to be displayed in the patient list 331a.

The controller 11 causes a request transmission button 331b corresponding to each patient ID to be displayed in the patient list 331a.

The request transmission button 331b is a button for transmitting a request to share the health record information of the patient to the user terminal 20 via the information management server 10.

The controller 11 causes the request transmission button 331b to be displayed for the patient ID associated with the health record information in the storage 14. On the other hand, the controller 11 does not cause the request transmission button 331b to be displayed for the patient ID not associated with the health record information in the storage 14.

That is, the controller 11 causes the request transmission button 331b (instructor) for accepting an instruction to transmit a data sharing request to be displayed in association with the patient ID (patient information) of the patient with whom the health record information (data) is associated.

Next, the controller 31 accepts a press operation of any of the request transmission buttons 331b by the medical professional via the operation part 32. Next, the controller 31 transmits, to the information management server 10, a request to share the health record information of the patient corresponding to the request transmission button 331b whose press operation has been accepted (step S2). The sharing request includes the facility ID of the medical facility having the medical facility terminal 30, the patient ID corresponding to the request transmission button 331b whose press operation has been accepted, and the like.

That is, the controller 31 transmits a request to share the health record information (data) from the medical facility terminal 30 used by a medical professional to the user terminal 20 used by a patient. The controller 31 functions as a transmitter.

As illustrated in FIG. 6, the request transmission button 331b is provided for each patient ID on the patient list screen 331. In this case, the controller 31 transmits a request to share all types of health record information of the patient corresponding to the request transmission button 331b whose press operation has been accepted. The types of the health record information are, for example, vital information (a blood glucose level, a blood pressure, a heart rate, a body temperature, and the like), medication information, mood information, a measurement result of a weight or the like, and the like.

The controller 31 may accept, from the medical professional via the operation part 32, a selection of the type of health record information for which a sharing request is to be transmitted. In this case, the controller 31 functions as a second acceptor. The controller 31 transmits a request to share the selected type of health record information.

Next, the controller 11 in the information management server 10 accepts the request to share the health record information of the patient transmitted from the medical facility terminal 30, thereby accepting the sharing request.

That is, the controller 11 accepts the request to share the health record information (data) from the medical facility terminal 30 to the user terminal 20, which is transmitted from the transmitter. The controller 11 functions as a first acceptor.

Next, the controller 11 transmits, to the user terminal 20 used by the patient indicated by the patient ID included in the sharing request, an approval screen for accepting approval for sharing the health record information (step S4).

The controller 21 in the user terminal 20 activates the health management app P1 on the basis of an operation by the patient via the operation part 22. Next, the controller 21 receives the approval screen from the information management server 10 via the health management app P1 and displays the received approval screen on the display 23 (step S5).

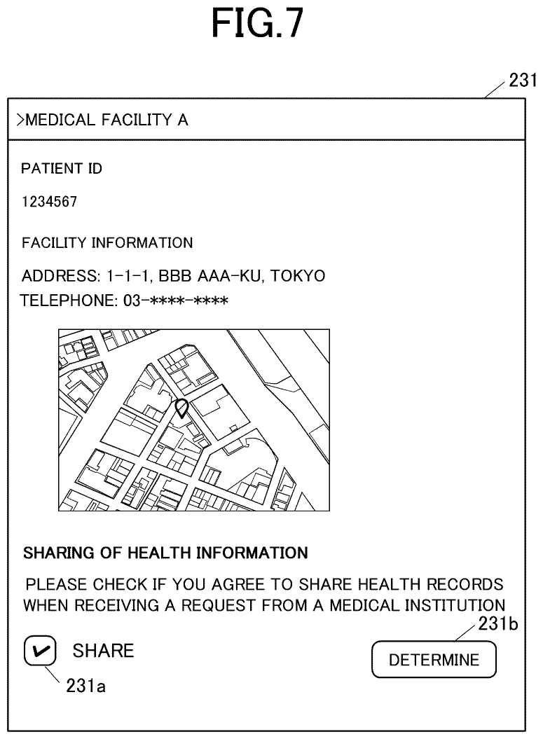

FIG. 7 illustrates an example of an approval screen 231 displayed on the display 23.

In the example illustrated in FIG. 7, the controller 11 causes the patient ID of the patient who uses the user terminal 20 to be displayed on the approval screen 231.

The controller 11 causes the facility information of the medical facility having the medical facility terminal 30 that has transmitted the request to share the health record information to be displayed on the approval screen 231. The controller 11 causes, for example, an address, a telephone number, and a map of a location of the medical facility to be displayed as the facility information.

The controller 11 causes an approval check box 231a to be displayed on the approval screen 231.

Next, in the approval check box 231a, the controller 21 accepts approval or rejection of the sharing of the health record information by the patient using the user terminal 20 (step S6). When the patient approves the sharing of the health record information, the patient checks the approval check box 231a. When the patient does not approve (rejects) the sharing of the health record information, the patient does not check the approval check box 231a.

That is, when accepting the request to share the health record information (data) from the medical facility terminal 30 to the user terminal 20, the controller 11 causes the display 23 included in the user terminal 20 to display an approval check box 231a (approver) for accepting approval for data sharing with the medical facility terminal 30.

The controller 21 accepts a press operation of a determination button 231b included in the approval screen 231 by the patient via the operation part 22.

When the controller 21 accepts the press operation of the determination button 231b by the patient, the controller 21 transmits approval or rejection information indicating approval or rejection of the sharing accepted in step S6 to the information management server 10 (step S7).

Next, the controller 11 in the information management server 10 receives the approval or rejection information transmitted from the user terminal 20. The controller 11 determines whether the patient has approved the sharing of the health record information on the basis of the received approval or rejection information (step S8).

When the patient has approved the sharing of the health record information (step S8; YES), the controller 11 transmits a display screen for displaying the health record information of the patient who has approved the sharing to the medical facility terminals 30 (step S9).

That is, when the approval of the sharing of the health record information (data) with the medical facility terminal 30 is accepted by the approval check box 231a (approver), the controller 11 shares the data with the medical facility terminals 30. The controller 11 functions as a sharer.

Next, the controller 31 in the medical facility terminal 30 receives the display screen for displaying the health record information of the patient transmitted from the information management server 10 via the medical facility app P2. Next, the controller 31 causes the display 33 to display a display screen for displaying the received health record information of the patient (step S10), and ends the information sharing process.

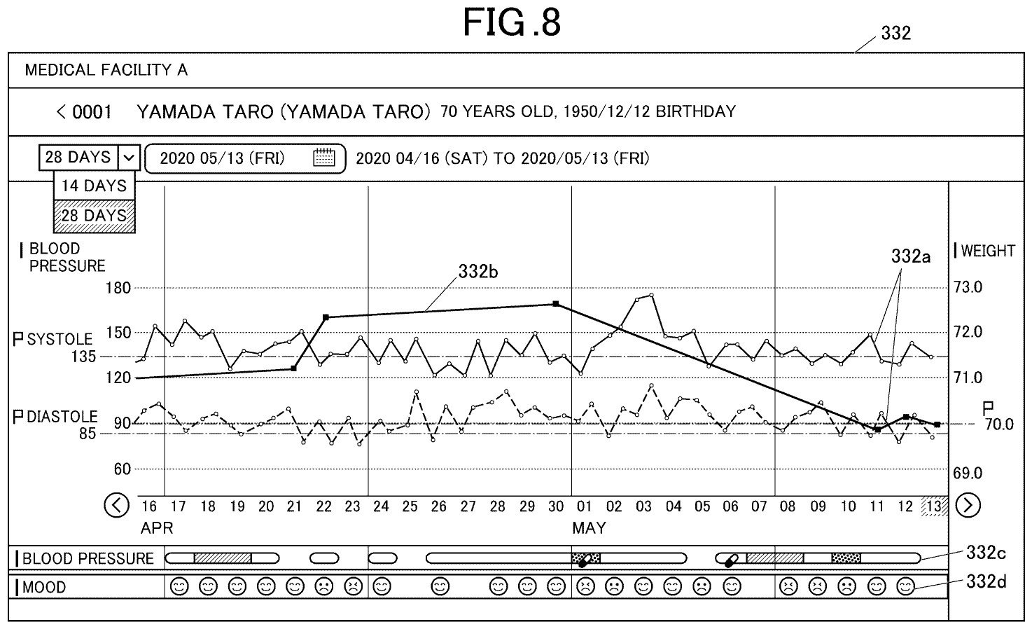

FIG. 8 illustrates an example of a display screen 332 for displaying the health record information of a patient.

In the example illustrated in FIG. 8, the controller 11 causes the health record information of the patient whose patient ID is “0001” to be displayed on the display screen 332.

As illustrated in FIG. 8, the controller 11 causes blood pressure information 332a to be displayed on the display screen 332 as the vital information for a predetermined period (e.g., Apr. 16, 2020 to May 13, 2020).

The controller 11 causes a weight measurement result 332b, medication information 332c, and mood information 332d of the patient to be displayed on the display screen 332 for a predetermined period (e.g., Apr. 16, 2020 to May 13, 2020).

On the other hand, when the patient has rejected the sharing of the health record information (step S8; NO), the controller 11 transmits notification information indicating that the patient has rejected the sharing of the health record information to the medical facility terminal 30 (step S11).

That is, when accepting that the sharing of the health record information (data) with the medical facility terminal 30 is not approved by the approval check box 231a (approver), the controller 11 notifies the medical facility terminal 30 that the sharing is not approved. The controller 11 functions as a notifier.

The controller 31 in the medical facility terminal 30 receives the notification information transmitted from the information management server 10. Next, the controller 31 displays, on the display 33, a message indicating that the patient has rejected the sharing of the health record information on the basis of the received notification information.

### [Others]

For example, in an emergency or the like, the patient can issue an instruction to share his/her health record information with the medical facility terminal 30 via the user terminal 20.

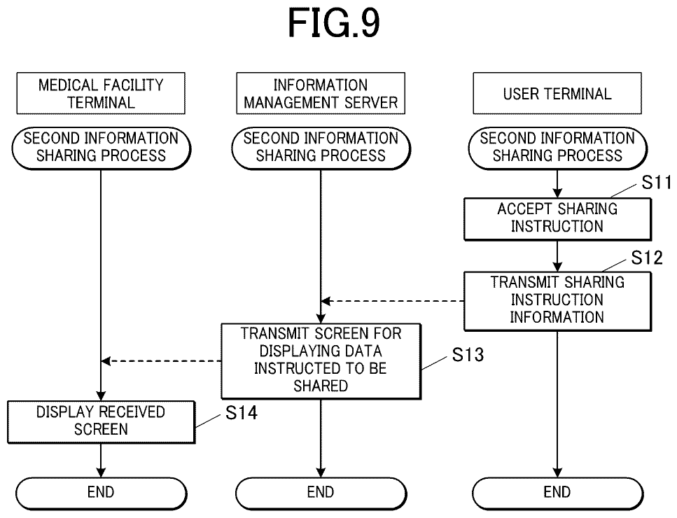

In this case, each device included in the information sharing system 100 executes a second information sharing process illustrated in FIG. 9.

### (Second Information Sharing Process)

When the patient wants to share the health record information with the medical facility terminal 30, the patient activates the health management app P1 via the operation part 22 in the user terminals 20.

When the health management app P1 is activated, the controller 21 in the user terminal 20 accepts an operation of instructing the sharing of the health record information with the medical facility device 30 by the patient via the operation part 22 (step S11).

That is, the controller 21 accepts, at the user terminal 20, an instruction to share the health record information (data) with the medical facility terminal 30. The controller 21 functions as a third acceptor.

Next, when accepting a sharing instruction operation by the patient, the controller 21 transmits sharing instruction information indicating the sharing instruction to the information management server 10 (step S12). The sharing instruction information includes the patient ID of the patient who uses the user terminal 20, the facility ID of the medical facility having the medical facility terminal 30 with which the health record information is shared, and the like.

Next, the controller 11 in the information management server 10 receives the sharing instruction information transmitted from the user terminal 20. Next, the controller 11 transmits a display screen for displaying the health record information of the patient to the medical facility terminals 30 on the basis of the received sharing instruction information (step S13). The medical facility terminal 30 is a terminal owned by the medical facility indicated by the facility ID included in the sharing instruction information. The patient is a patient indicated by the patient ID included in the sharing instruction information.

That is, when the third acceptor accepts an instruction to share the health record information (data) with the medical facility terminal 30, the controller 11 shares the data with the medical facility terminal 30.

The controller 31 in the medical facility terminal 30 activates the medical facility app P2 on the basis of a patient's operation via the operation part 32. Next, the controller 31 receives, via the medical facility app P2, a display screen for displaying the health record information of the patient transmitted from the information management server 10. Next, the controller 31 displays a display screen for displaying the received health record information of the patient on the display 33 (step S14), and ends the information sharing process.

### [Effect]

As described above, the information sharing system 100 of the present embodiment includes the transmitter (controller 31) that transmits the data sharing request from the medical facility terminal 30 used by the medical professional to the user terminal 20 used by the patient.

The information sharing system 100 of the present embodiment includes a first acceptor (controller 11) that accepts the sharing request transmitted from the transmitter.

The information sharing system 100 of the present embodiment includes the controller 11 that, when the first acceptor has accepted the sharing request, causes the display 23 included in the user terminal 20 to display an approver (approval check box 231a) for accepting approval for data sharing with the medical facility terminal 30.

The information sharing system 100 of the present embodiment includes a sharer (controller 11) that shares data with the medical facility terminal 30 when the approval for sharing of data with the medical facility terminal 30 is accepted by the approver.

Thus, on the basis of the patient's intention to approve or reject the sharing of the data which is the health record information registered via the health management app P1, the doctor can view the data of the patient who has accepted the sharing request. That is, the doctor can appropriately view the data of the patient.

In the information sharing system 100 of the present embodiment, the approver (approval check box 231a) can accept that the sharing of the data with the medical facility terminal 30 is not approved.

The information sharing system 100 of the present embodiment includes a notifier (controller 11) that notifies the medical facility terminal 30 that the data sharing with the medical facility terminal 30 has not been approved when the approver (approval check box 231a) accepts that the sharing of the data with the medical facility terminal 30 is not approved.

Thus, the doctor can easily recognize that the patient has rejected the data sharing.

In the information sharing system 100 of the present embodiment, the controller 11 causes the instructor (request transmission button 331b) for accepting an instruction to transmit a data sharing request to be displayed in association with the patient information of the patient with whom the data is associated.

Thus, the doctor can easily recognize the patient ID associated with the data (health record information). That is, the doctor can easily recognize the patient whose health record information is recorded.

The information sharing system 100 of the present embodiment includes a second acceptor (controller 31) that accepts selection of a data type.

In the information sharing system 100 of the present embodiment, the transmitter (controller 31) transmits a request to share the data of the type the selection of which has been accepted by the second acceptor.

Thus, the doctor can transmit a request to share a desired type of data.

The information sharing system 100 of the present embodiment includes a third acceptor (controller 21) that accepts, at the user terminal 20, an instruction to share data with the medical facility terminal 30.

In the information sharing system 100 of the present embodiment, when the third acceptor has accepted the instruction, the sharer (controller 11) shares the data with the medical facility terminal 30.

Thus, for example, in an emergency or the like, the patient can share his/her own data (health record information) with the medical facility terminal 30.

Although the embodiments of the present invention have been described above, the present invention is not limited to the embodiments, and it is needless to say that various modifications can be made without departing from the scope of the present invention.

For example, in the information sharing system 100 of the above-described embodiment, one medical facility terminal 30 is used in one medical facility, but the present invention is not limited thereto. A plurality of medical facility terminals 30 may be also used in one medical facility.

Besides, the detailed configuration and detailed operation of each part constituting the dose management system can also be appropriately modified without departing from the spirit and scope of the present invention.

Although embodiments of the present invention have been described and illustrated in detail, the disclosed embodiments are made for purposes of illustration and example only and not limitation. The scope of the present invention should be interpreted by terms of the appended claims.

The entire disclosure of Japanese Patent Application No. 2023-183992 filed on Oct. 26, 2023 is incorporated herein by reference in its entirety.

What is claimed is:

1. An information sharing system, comprising:
a first hardware processor that transmits a data sharing request from a medical facility terminal used by a medical professional to a user terminal used by a patient; and
a second hardware processor that accepts the sharing request transmitted from the first hardware processor, wherein
the second hardware processor, upon acceptance of the sharing request, causes a display included in the user terminal to display an approver for accepting approval for sharing the data with the medical facility terminal, and upon acceptance of the approval for sharing the data with the medical facility terminal by the approver, causes the data to be shared with the medical facility terminal.

2. The information sharing system according to claim 1, wherein the approver is capable of accepting that the sharing of the data with the medical facility terminal is not approved.

3. The information sharing system according to claim 2, wherein, the second hardware processor notifies the medical facility terminal that the sharing of the data with medical facility terminal has not been approved when the approver accepts that the sharing of the data with the medical facility terminal is not accepted.

4. The information sharing system according to claim 1, wherein the second hardware processor causes an instructor for accepting an instruction to transmit the sharing request of the data to be displayed in association with patient information of a patient with whom the data is associated.

5. The information sharing system according to claim 1, wherein the first hardware processor accepts a selection of a type of the data, and transmits the sharing request of the data of the selected type.

6. The information sharing system according to claim 1, further comprising:
a third hardware processor that accepts, at the user terminal, an instruction to share the data with the medical facility terminal, wherein
the second hardware processor shares the data with the medical facility terminal when the instruction is accepted by the third hardware processor.

7. A program causing a computer of an information sharing system to execute:
accepting a data sharing request from a medical facility terminal used by a medical professional to a user terminal used by a patient;
displaying an approver for accepting approval for sharing the data with the medical facility terminal on a display included in the user terminal upon acceptance of the sharing request; and
sharing the data with the medical facility terminal upon acceptance of the approval for sharing the data with the medical facility terminal by the approver.
